(19) United States
(12) Patent Application Publication
(10) Pub. No.: US 2022/0280084 A1
(43) Pub. Date: Sep. 8, 2022

(54) Presentation of Graphical Content Associated With Measured Brain Activity

(71) Applicant: HI LLC, Los Angeles, CA (US)

(72) Inventors: Bryan Johnson, Culver City, CA (US); Ryan Field, Culver City, CA (US); Katherine Perdue, Los Angeles, CA (US)

(21) Appl. No.: 17/559,316

(22) Filed: Dec. 22, 2021

Related U.S. Application Data
(60) Provisional application No. 63/156,785, filed on Mar. 4, 2021.

(51) Int. Cl. A61B 5/16; A61B 5/00; A61B 5/25

(52) U.S. Cl. CPC A61B 5/165; A61B 5/6803; A61B 5/7475; A61B 5/25

(57) ABSTRACT

An illustrative system includes a brain interface system configured to be worn by a user and to output brain activity data representative of brain activity of the user and a computing device configured to obtain the brain activity data, determine, based on the brain activity data, a characteristic of the user, and present, by way of a graphical user interface, graphical content representative of the characteristic.

### RELATED APPLICATIONS

The present application claims priority under 35 U.S.C. § 119(e) to U.S. Provisional Patent Application No. 63/156,785, filed Mar. 4, 2021, and incorporated herein by reference in its entirety.

### BACKGROUND INFORMATION

A person's brain may be affected by a variety of different factors. For example, a person's brain may be fatigued, stimulated, engaged, stressed, and/or otherwise affected by things that the person sees, hears, and/or does, environmental conditions, actions taken by others, and/or a variety of other inputs. Measurement of brain activity may provide insight into how the brain is affected by these factors. However, it may be difficult for many people to interpret measured brain activity in a way that is meaningful and actionable.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 shows an exemplary configuration that includes a brain interface system, a computing device, and a display.

FIGS. 2-4, 5A and 5B show various optical measurement systems that may implement the brain interface system shown in FIG. 1.

FIGS. 6-7 show various multimodal measurement systems that may implement the brain interface system shown in FIG. 1.

FIG. 8 shows an exemplary magnetic field measurement system that may implement the brain interface system shown in FIG. 1.

FIG. 9 shows an illustrative configuration in which a computing device is configured to implement a machine learning model to determine a characteristic of a user.

FIGS. 10-16 show examples of presenting graphical content associated with a determined user characteristic.

FIGS. 17-19 show configurations in which a computing device is configured to output, based on a determined user characteristic, control data.

FIG. 20 shows an illustrative configuration in which a computing device is configured to access both brain activity data and sensor data output by a sensor.

FIG. 21 illustrates an exemplary method.

FIG. 22 illustrates an exemplary computing device.

### DETAILED DESCRIPTION

Various ways in which graphical content associated with brain activity as measured by a non-invasive brain interface system may be presented are described herein. For example, an illustrative system may include a brain interface system and a computing device. The brain interface system may be configured to be worn by a user and to output brain activity data representative of brain activity of the user. The computing device may be configured to obtain the brain activity data, determine, based on the brain activity data, a characteristic of the user, and present, by way of a graphical user interface, graphical content representative of the characteristic.

The embodiments described herein may transform complex and difficult to understand brain activity data into relatively easy to understand graphical content that can be used by the user and/or other personnel to take various actions that may improve brain health, help the user perform one or more tasks in a more optimal manner, prevent long term damage to the brain, achieve a desired mental state, and/or otherwise understand how the brain is functioning. These and other benefits are described more fully herein.

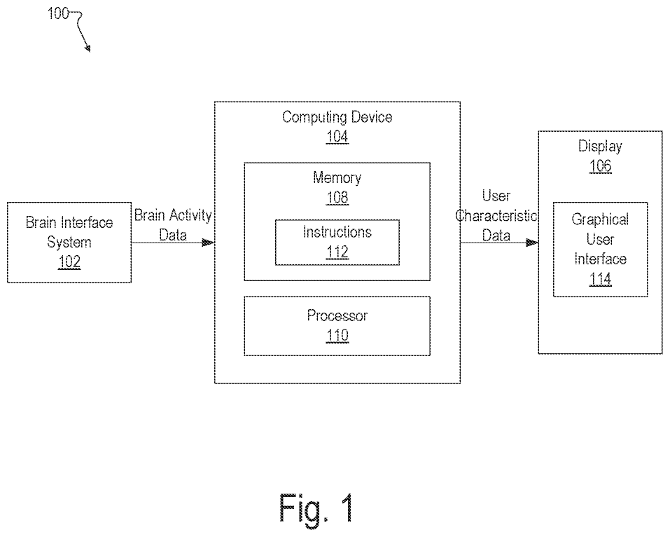

FIG. 1 shows an exemplary configuration 100 that includes a brain interface system 102, a computing device 104, and a display 106.

Brain interface system 102 may be configured to be worn by a user and to output brain activity data representative of brain activity of the user while the brain interface system 102 is being worn by the user. As described herein, the brain activity data may include any data output by any of the implementations of brain interface system 102 described herein. For example, the brain activity data may include or be based on optical-based, electrical-based, and/or magnetic field-based measurements of activity within the brain, as described herein.

Computing device 104 may be configured to obtain (e.g., receive or otherwise access) the brain activity data. This may be performed in any suitable manner. For example, computing device 104 may receive the brain activity data from brain interface system 102 by way a wired and/or wireless (e.g., Bluetooth, WiFi, etc.) connection.

Computing device 104 may be further configured to determine, based on the brain activity data, one or more characteristics of the user. This may be performed in any suitable manner, examples of which are described herein.

In some examples, as shown, computing device 104 may generate user characteristic data representative of the one or more characteristics. Example characteristics of the user that computing device 104 may determine based on brain activity data are described herein.

Computing device 104 may be implemented by one or more computing or processing devices, such as one or more personal computers, mobile devices (e.g., a mobile phone, a tablet computer, etc.), servers, and/or any other type of computing device as may serve a particular implementation. In some examples, computing device 104 may be included in brain interface system 102. Additionally or alternatively, computing device 104 may be separate from (i.e., remote from and communicatively coupled to) brain interface system 102.

As shown, computing device 104 may include memory 108 and a processor 110. Computing device 104 may include additional or alternative components as may serve a particular implementation. Each component may be implemented by any suitable combination of hardware and/or software.

Memory 108 may maintain (e.g., store) executable data used by processor 110 to perform one or more of the operations described herein as being performed by computing device 104. For example, memory 108 may store instructions 112 that may be executed by processor 110 to generate game control data and/or perform one or more operations based on the game control data. Instructions 112 may be implemented by any suitable application, program, software, code, and/or other executable data instance. Memory 108 may also maintain any data received, generated, managed, used, and/or transmitted by processor 110.

Processor 110 may be configured to perform (e.g., execute instructions 112 stored in memory 108 to perform) various operations described herein as being performed by computing device 104. Examples of such operations are described herein.

Display 106 may be implemented by any suitable display device configured to display a graphical user interface 114. In some examples, display 106 may be integrated into computing device 104. Alternatively, display 106 may be a standalone display (e.g., a monitor) connected to computing device 104.

As described herein, computing device 104 may be configured to present, by way of graphical user interface 114, graphical content representative of the one or more characteristics represented by the user characteristic data generated by computing device 104.

In some examples, computing device 104 may obtain the brain activity data, determine the one or more characteristics, and present the graphical content in substantially real time while brain interface system 102 outputs the brain activity data. In this manner, the graphical content may be displayed and acted upon while the user is wearing and using the brain interface system 102.

As used herein, “real time” and “substantially real time” and “concurrently” will be understood to relate to data processing and/or other actions that are performed immediately, as well as conditions and/or circumstances that are accounted for as they exist in the moment, or at the same time, when the processing or other actions are performed. For example, a real-time operation may refer to an operation that is performed immediately and without undue delay, even if it is not possible for there to be absolutely zero delay. Similarly, real-time data, real-time representations, real-time conditions, at the same time conditions, and so forth, will be understood to refer to data, representations, and conditions that relate to a present moment in time or a moment in time when decisions are being made and operations are being performed (e.g., even if after a short delay), such that the data, representations, conditions, and so forth are temporally relevant to the decisions being made and/or the operations being performed.

Additionally or alternatively, computing device 104 may determine the user characteristic and present the graphical content subsequent to when brain interface system 102 outputs the brain activity data. For example, the brain activity data may be stored and then processed by computing device 104 days after brain interface system 102 outputs the brain activity data.

Brain interface system 102 may be implemented by any suitable wearable non-invasive brain interface system as may serve a particular implementation. For example, brain interface system 102 may be implemented by a wearable optical measurement system configured to perform optical-based brain data acquisition operations, such as any of the wearable optical measurement systems described in U.S. patent application Ser. No. 17/176,315, filed Feb. 16, 2021 and published as US2021/0259638A1; U.S. patent application Ser. No. 17/176,309, filed Feb. 16, 2021 and published as US2021/0259614A1; U.S. patent application Ser. No. 17/176,460, filed Feb. 16, 2021, issued as U.S. Pat. No. 11,096,620; U.S. patent application Ser. No. 17/176,470, filed Feb. 16, 2021 and published as US2021/0259619A1; U.S. patent application Ser. No. 17/176,487, filed Feb. 16, 2021 and published as US2021/0259632A1, U.S. patent application Ser. No. 17/176,539, filed Feb. 16, 2021 and published as US2021/0259620A1; U.S. patent application Ser. No. 17/176,560, filed Feb. 16, 2021 and published as US2021/0259597A1; U.S. patent application Ser. No. 17/176,466, filed Feb. 16, 2021 and published as US2021/0263320A1; and Han Y. Ban, et al., “Kernel Flow: A High Channel Count Scalable TD-fNIRS System,” SPIE Photonics West Conference (Mar. 6, 2021), which applications and publications are incorporated herein by reference in their entirety.

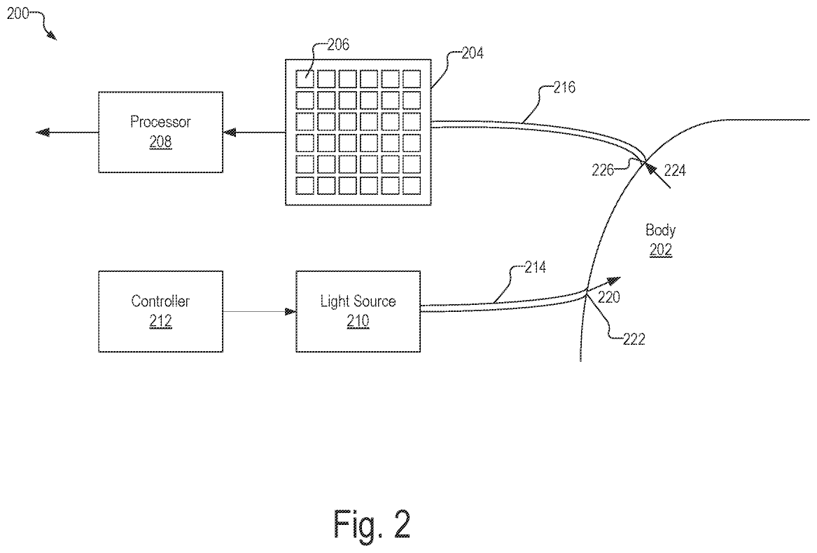

To illustrate, FIGS. 2-4, 5A, and 5B, show various optical measurement systems and related components that may implement brain interface system 102. The optical measurement systems described herein are merely illustrative of the many different optical-based brain interface systems that may be used in accordance with the systems and methods described herein.

FIG. 2 shows an optical measurement system 200 that may be configured to perform an optical measurement operation with respect to a body 202 (e.g., the brain). Optical measurement system 200 may, in some examples, be portable and/or wearable by a user.

In some examples, optical measurement operations performed by optical measurement system 200 are associated with a time domain-based optical measurement technique. Example time domain-based optical measurement techniques include, but are not limited to, time-correlated single-photon counting (TCSPC), time domain near infrared spectroscopy (TD-NIRS), time domain diffusive correlation spectroscopy (TD-DCS), and time domain digital optical tomography (TD-DOT).

Optical measurement system 200 (e.g., an optical measurement system that is implemented by a wearable device or other configuration, and that employs a time domain-based (e.g., TD-NIRS) measurement technique) may detect blood oxygenation levels and/or blood volume levels by measuring the change in shape of laser pulses after they have passed through target tissue, e.g., brain, muscle, finger, etc. As used herein, a shape of laser pulses refers to a temporal shape, as represented for example by a histogram generated by a time-to-digital converter (TDC) coupled to an output of a photodetector, as will be described more fully below.

As shown, optical measurement system 200 includes a detector 204 that includes a plurality of individual photodetectors (e.g., photodetector 206), a processor 208 coupled to detector 204, a light source 210, a controller 212, and optical conduits 214 and 216 (e.g., light pipes). However, one or more of these components may not, in certain embodiments, be considered to be a part of optical measurement system 200. For example, in implementations where optical measurement system 200 is wearable by a user, processor 208 and/or controller 212 may in some embodiments be separate from optical measurement system 200 and not configured to be worn by the user.

Detector 204 may include any number of photodetectors 206 as may serve a particular implementation, such as 2ⁿ photodetectors (e.g., 256, 512, . . . , 26384, etc.), where n is an integer greater than or equal to one (e.g., 4, 5, 8, 20, 21, 24, etc). Photodetectors 206 may be arranged in any suitable manner.

Photodetectors 206 may each be implemented by any suitable circuit configured to detect individual photons of light incident upon photodetectors 206. For example, each photodetector 206 may be implemented by a single photon avalanche diode (SPAD) circuit and/or other circuitry as may serve a particular implementation. The SPAD circuit may be gated in any suitable manner or be configured to operate in a free running mode with passive quenching. For example, photodetectors 206 may be configured to operate in a free-running mode such that photodetectors 206 are not actively armed and disarmed (e.g., at the end of each predetermined gated time window). In contrast, while operating in the free-running mode, photodetectors 206 may be configured to reset within a configurable time period after an occurrence of a photon detection event (i.e., after photodetector 206 detects a photon) and immediately begin detecting new photons. However, only photons detected within a desired time window (e.g., during each gated time window) may be included in the histogram that represents a light pulse response of the target (e.g., a temporal point spread function (TPSF)). The terms histogram and TPSF are used interchangeably herein to refer to a light pulse response of a target.

Processor 208 may be implemented by one or more physical processing (e.g., computing) devices. In some examples, processor 208 may execute instructions (e.g., software) configured to perform one or more of the operations described herein.

Light source 210 may be implemented by any suitable component configured to generate and emit light. For example, light source 210 may be implemented by one or more laser diodes, distributed feedback (DFB) lasers, super luminescent diodes (SLDs), light emitting diodes (LEDs), diode-pumped solid-state (DPSS) lasers, super luminescent light emitting diodes (sLEDs), vertical-cavity surface-emitting lasers (VCSELs), titanium sapphire lasers, micro light emitting diodes (mLEDs), and/or any other suitable laser or light source. In some examples, the light emitted by light source 210 is high coherence light (e.g., light that has a coherence length of at least 5 centimeters) at a predetermined center wavelength.

Light source 210 is controlled by controller 212, which may be implemented by any suitable computing device (e.g., processor 208), integrated circuit, and/or combination of hardware and/or software as may serve a particular implementation. In some examples, controller 212 is configured to control light source 210 by turning light source 210 on and off and/or setting an intensity of light generated by light source 210. Controller 212 may be manually operated by a user, or may be programmed to control light source 210 automatically.

Light emitted by light source 210 may travel via an optical conduit 214 (e.g., a light pipe, a single-mode optical fiber, and/or or a multi-mode optical fiber) to body 202 of a subject. Body 202 may include any suitable turbid medium. For example, in some implementations, body 202 is a brain or any other body part of a human or other animal. Alternatively, body 202 may be a non-living object. For illustrative purposes, it will be assumed in the examples provided herein that body 202 is a human brain.

As indicated by arrow 220, the light emitted by light source 210 enters body 202 at a first location 222 on body 202. Accordingly, a distal end of optical conduit 214 may be positioned at (e.g., right above, in physical contact with, or physically attached to) first location 222 (e.g., to a scalp of the subject). In some examples, the light may emerge from optical conduit 214 and spread out to a certain spot size on body 202 to fall under a predetermined safety limit. At least a portion of the light indicated by arrow 220 may be scattered within body 202.

As used herein, “distal” means nearer, along the optical path of the light emitted by light source 210 or the light received by detector 204, to the target (e.g., within body 202) than to light source 210 or detector 204. Thus, the distal end of optical conduit 214 is nearer to body 202 than to light source 210, and the distal end of optical conduit 216 is nearer to body 202 than to detector 204. Additionally, as used herein, “proximal” means nearer, along the optical path of the light emitted by light source 210 or the light received by detector 204, to light source 210 or detector 204 than to body 202. Thus, the proximal end of optical conduit 214 is nearer to light source 210 than to body 202, and the proximal end of optical conduit 216 is nearer to detector 204 than to body 202.

As shown, the distal end of optical conduit 216 (e.g., a light pipe, a light guide, a waveguide, a single-mode optical fiber, and/or a multi-mode optical fiber) is positioned at (e.g., right above, in physical contact with, or physically attached to) output location 226 on body 202. In this manner, optical conduit 216 may collect at least a portion of the scattered light (indicated as light 224) as it exits body 202 at location 226 and carry light 224 to detector 204. Light 224 may pass through one or more lenses and/or other optical elements (not shown) that direct light 224 onto each of the photodetectors 206 included in detector 204. In cases where optical conduit 216 is implemented by a light guide, the light guide may be spring loaded and/or have a cantilever mechanism to allow for conformably pressing the light guide firmly against body 202.

Photodetectors 206 may be connected in parallel in detector 204. An output of each of photodetectors 206 may be accumulated to generate an accumulated output of detector 204. Processor 208 may receive the accumulated output and determine, based on the accumulated output, a temporal distribution of photons detected by photodetectors 206. Processor 208 may then generate, based on the temporal distribution, a histogram representing a light pulse response of a target (e.g., brain tissue, blood flow, etc.) in body 202. Such a histogram is illustrative of the various types of brain activity measurements that may be performed by brain interface system 102.

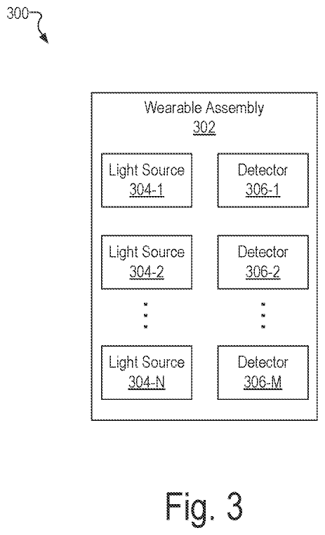

FIG. 3 shows an exemplary optical measurement system 300 in accordance with the principles described herein. Optical measurement system 300 may be an implementation of optical measurement system 200 and, as shown, includes a wearable assembly 302, which includes N light sources 304 (e.g., light sources 304-1 through 304-N) and M detectors 306 (e.g., detectors 306-1 through 306-M). Optical measurement system 300 may include any of the other components of optical measurement system 200 as may serve a particular implementation. N and M may each be any suitable value (i.e., there may be any number of light sources 304 and detectors 306 included in optical measurement system 300 as may serve a particular implementation).

Light sources 304 are each configured to emit light (e.g., a sequence of light pulses) and may be implemented by any of the light sources described herein. Detectors 306 may each be configured to detect arrival times for photons of the light emitted by one or more light sources 304 after the light is scattered by the target. For example, a detector 306 may include a photodetector configured to generate a photodetector output pulse in response to detecting a photon of the light and a time-to-digital converter (TDC) configured to record a timestamp symbol in response to an occurrence of the photodetector output pulse, the timestamp symbol representative of an arrival time for the photon (i.e., when the photon is detected by the photodetector).

Wearable assembly 302 may be implemented by any of the wearable devices, modular assemblies, and/or wearable units described herein. For example, wearable assembly 302 may be implemented by a wearable device (e.g., headgear) configured to be worn on a user's head. Wearable assembly 302 may additionally or alternatively be configured to be worn on any other part of a user's body.

Optical measurement system 300 may be modular in that one or more components of optical measurement system 300 may be removed, changed out, or otherwise modified as may serve a particular implementation. As such, optical measurement system 300 may be configured to conform to three-dimensional surface geometries, such as a user's head. Exemplary modular optical measurement systems comprising a plurality of wearable modules are described in more detail in one or more of the patent applications incorporated herein by reference.

Fla 4 shows an illustrative modular assembly 400 that may implement optical measurement system 300. Modular assembly 400 is illustrative of the many different implementations of optical measurement system 300 that may be realized in accordance with the principles described herein.

As shown, modular assembly 400 includes a plurality of modules 402 (e.g., modules 402-1 through 402-3) physically distinct one from another. While three modules 402 are shown to be included in modular assembly 400, in alternative configurations, any number of modules 402 (e.g., a single module up to sixteen or more modules) may be included in modular assembly 400.

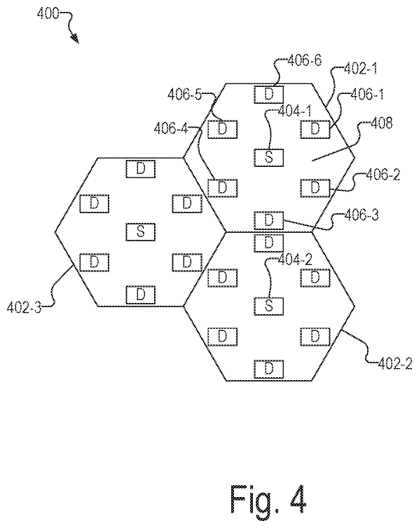

Each module 402 includes a light source (e.g., light source 404-1 of module 402-1 and light source 404-2 of module 402-2) and a plurality of detectors (e.g., detectors 406-1 through 406-6 of module 402-1). In the particular implementation shown in FIG. 4, each module 402 includes a single light source and six detectors. Each light source is labeled “S” and each detector is labeled “D”.

Each light source depicted in FIG. 4 may be implemented by one or more light sources similar to light source 210 and may be configured to emit light directed at a target (e.g., the brain).

Each light source depicted in FIG. 4 may be located at a center region of a surface of the light sources corresponding module. For example, light source 404-1 is located at a center region of a surface 408 of module 402-1. In alternative implementations, a light source of a module may be located away from a center region of the module.

Each detector depicted in FIG. 4 may implement or be similar to detector 204 and may include a plurality of photodetectors (e.g., SPADs) as well as other circuitry (e.g., TDCs), and may be configured to detect arrival times for photons of the light emitted by one or more light sources after the light is scattered by the target.

The detectors of a module may be distributed around the light source of the module. For example, detectors 406 of module 402-1 are distributed around light source 404-1 on surface 408 of module 402-1. In this configuration, detectors 406 may be configured to detect photon arrival times for photons included in light pulses emitted by light source 404-1. In some examples, one or more detectors 406 may be close enough to other light sources to detect photon arrival times for photons included in light pulses emitted by the other light sources. For example, because detector 406-3 is adjacent to module 402-2, detector 406-3 may be configured to detect photon arrival times for photons included in light pulses emitted by light source 404-2 (in addition to detecting photon arrival times for photons included in light pulses emitted by light source 404-1).

In some examples, the detectors of a module may all be equidistant from the light source of the same module. In other words, the spacing between a light source (i.e., a distal end portion of a light source optical conduit) and the detectors (i.e., distal end portions of optical conduits for each detector) are maintained at the same fixed distance on each module to ensure homogeneous coverage over specific areas and to facilitate processing of the detected signals. The fixed spacing also provides consistent spatial (lateral and depth) resolution across the target area of interest, e.g., brain tissue. Moreover, maintaining a known distance between the light source, e.g., light emitter, and the detector allows subsequent processing of the detected signals to infer spatial (e.g., depth localization, inverse modeling) information about the detected signals. Detectors of a module may be alternatively disposed on the module as may serve a particular implementation.

In some examples, modular assembly 400 can conform to a three-dimensional (3D) surface of the human subject's head, maintain tight contact of the detectors with the human subject's head to prevent detection of ambient light, and maintain uniform and fixed spacing between light sources and detectors. The wearable module assemblies may also accommodate a large variety of head sizes, from a young child's head size to an adult head size, and may accommodate a variety of head shapes and underlying cortical morphologies through the conformability and scalability of the wearable module assemblies. These exemplary modular assemblies and systems are described in more detail in U.S. patent application Ser. Nos. 17/176,470; 17/176,487; 17/176,539; 17/176,560; 17/176,460; and 17/176,466, which applications have been previously incorporated herein by reference in their respective entireties.

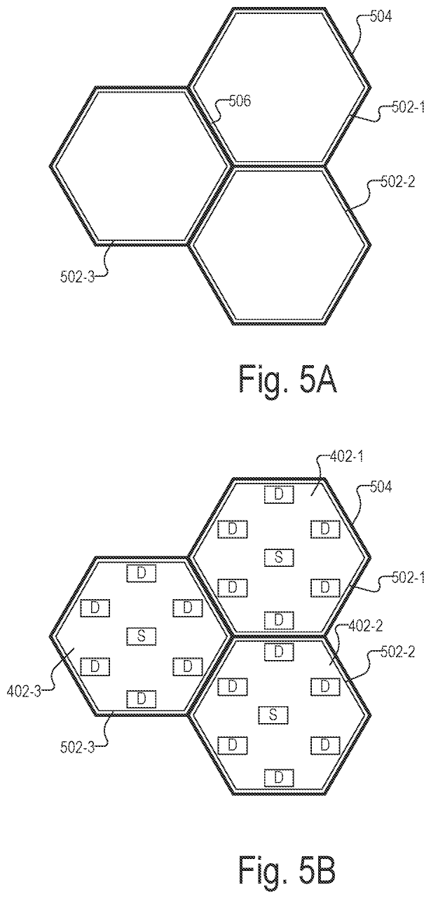

In FIG. 4, modules 402 are shown to be adjacent to and touching one another. Modules 402 may alternatively be spaced apart from one another. For example, FIGS. 5A-5B show an exemplary implementation of modular assembly 400 in which modules 402 are configured to be inserted into individual slots 502 (e.g., slots 502-1 through 502-3, also referred to as cutouts) of a wearable assembly 504. In particular, FIG. 5A shows the individual slots 502 of the wearable assembly 504 before modules 402 have been inserted into respective slots 502, and FIG. 5B shows wearable assembly 504 with individual modules 402 inserted into respective individual slots 502.

Wearable assembly 504 may implement wearable assembly 302 and may be configured as headgear and/or any other type of device configured to be worn by a user.

As shown in FIG. 5A, each slot 502 is surrounded by a wall (e.g., wall 506) such that when modules 402 are inserted into their respective individual slots 502, the walls physically separate modules 402 one from another. In alternative embodiments, a module (e.g., module 402-1) may be in at least partial physical contact with a neighboring module (e.g., module 402-2).

Each of the modules described herein may be inserted into appropriately shaped slots or cutouts of a wearable assembly, as described in connection with FIGS. 5A-5B. However, for ease of explanation, such wearable assemblies are not shown in the figures.

As shown in FIGS. 4 and 5B, modules 402 may have a hexagonal shape. Modules 402 may alternatively have any other suitable geometry (e.g., in the shape of a pentagon, octagon, square, rectangular, circular, triangular, free-form, etc.).

As another example, brain interface system 102 may be implemented by a wearable multimodal measurement system configured to perform both optical-based brain data acquisition operations and electrical-based brain data acquisition operations, such as any of the wearable multimodal measurement systems described in U.S. patent application Ser. Nos. 17/176,315 and 17/176,309, which applications have been previously incorporated herein by reference in their respective entireties.

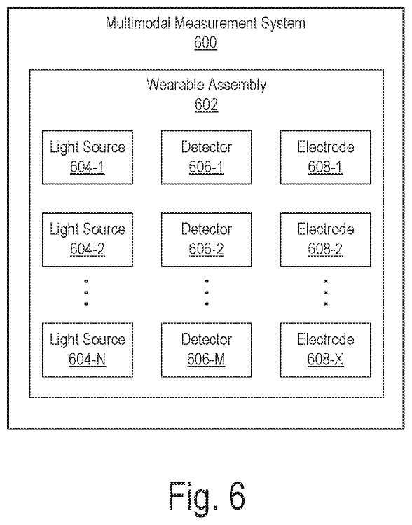

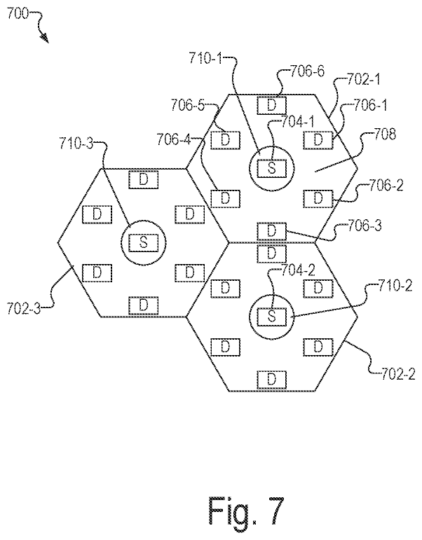

To illustrate, FIGS. 6-7 show various multimodal measurement systems that may implement brain interface system 102. The multimodal measurement systems described herein are merely illustrative of the many different multimodal-based brain interface systems that may be used in accordance with the systems and methods described herein.

FIG. 6 shows an exemplary multimodal measurement system 600 in accordance with the principles described herein. Multimodal measurement system 600 may at least partially implement optical measurement system 200 and, as shown, includes a wearable assembly 602 (which is similar to wearable assembly 302), which includes N light sources 604 (e.g., light sources 604-1 through 604-N, which are similar to light sources 304), M detectors 606 (e.g., detectors 606-1 through 606-M, which are similar to detectors 306), and X electrodes (e.g., electrodes 608-1 through 608-X). Multimodal measurement system 600 may include any of the other components of optical measurement system 200 as may serve a particular implementation. N, M, and X may each be any suitable value (i.e., there may be any number of light sources 604, any number of detectors 606, and any number of electrodes 608 included in multimodal measurement system 600 as may serve a particular implementation).

Electrodes 608 may be configured to detect electrical activity within a target (e.g., the brain). Such electrical activity may include electroencephalogram (EEG) activity and/or any other suitable type of electrical activity as may serve a particular implementation. In some examples, electrodes 608 are all conductively coupled to one another to create a single channel that may be used to detect electrical activity. Alternatively, at least one electrode included in electrodes 608 is conductively isolated from a remaining number of electrodes included in electrodes 608 to create at least two channels that may be used to detect electrical activity.

FIG. 7 shows an illustrative modular assembly 700 that may implement multimodal measurement system 600. As shown, modular assembly 700 includes a plurality of modules 702 (e.g., modules 702-1 through 702-3). While three modules 702 are shown to be included in modular assembly 700, in alternative configurations, any number of modules 702 (e.g., a single module up to sixteen or more modules) may be included in modular assembly 700. Moreover, while each module 702 has a hexagonal shape, modules 702 may alternatively have any other suitable geometry (e.g., in the shape of a pentagon, octagon, square, rectangular, circular, triangular, free-form, etc.).

Each module 702 includes a light source (e.g., light source 704-1 of module 702-1 and light source 704-2 of module 702-2) and a plurality of detectors (e.g., detectors 706-1 through 706-6 of module 702-1). In the particular implementation shown in FIG. 7, each module 702 includes a single light source and six detectors. Alternatively, each module 702 may have any other number of light sources (e.g., two light sources) and any other number of detectors. The various components of modular assembly 700 shown in FIG. 7 are similar to those described in connection with FIG. 4.

As shown, modular assembly 700 further includes a plurality of electrodes 710 (e.g., electrodes 710-1 through 710-3), which may implement electrodes 608. Electrodes 710 may be located at any suitable location that allows electrodes 710 to be in physical contact with a surface (e.g., the scalp and/or skin) of a body of a user. For example, in modular assembly 700, each electrode 710 is on a module surface configured to face a surface of a user's body when modular assembly 700 is worn by the user. To illustrate, electrode 710-1 is on surface 708 of module 702-1. Moreover, in modular assembly 700, electrodes 710 are located in a center region of each module 702 and surround each module's light source 704. Alternative locations and configurations for electrodes 710 are possible.

As another example, brain interface system 102 may be implemented by a wearable magnetic field measurement system configured to perform magnetic field-based brain data acquisition operations, such as any of the magnetic field measurement systems described in U.S. patent application Ser. No. 16/862,879, filed Apr. 30, 2020 and published as US2020/0348368A1; U.S. Provisional Application No. 63/170,892, filed Apr. 5, 2021, U.S. Non-Provisional application Ser. No. 17/338,429, filed Jun. 3, 2021, and Ethan J. Pratt, et al., “Kernel Flux: A Whole-Head 432-Magnetometer Optically-Pumped Magnetoencephalography (OP-MEG) System for Brain Activity Imaging During Natural Human Experiences,” SPIE Photonics West Conference (Mar. 6, 2021), which applications and publications are incorporated herein by reference in their entirety. In some examples, any of the magnetic field measurement systems described herein may be used in a magnetically shielded environment which allows for natural user movement as described for example in U.S. Provisional Patent Application No. 63/076,015, filed Sep. 9, 2020, and U.S. Non-Provisional patent application Ser. No. 17/328,235, filed May 24, 2021 and published as US2021/0369166A1, which applications are incorporated herein by reference in their entirety.

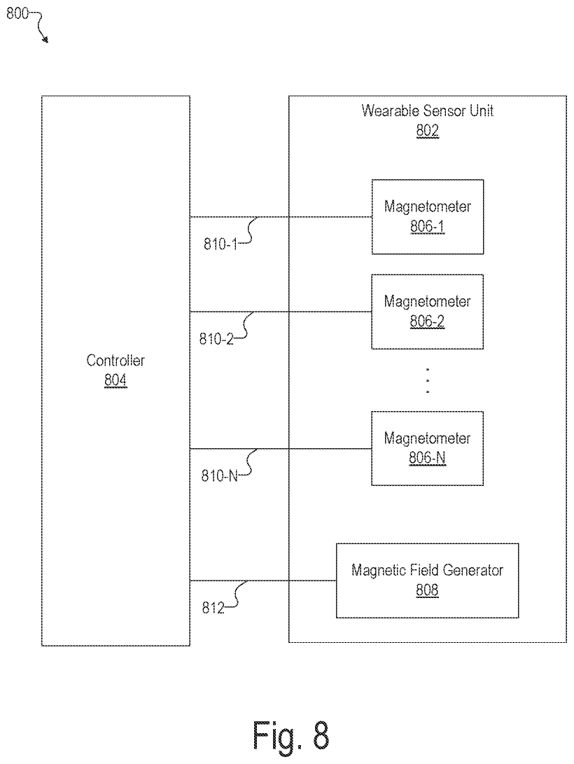

FIG. 8 shows an exemplary magnetic field measurement system 800 (“system 800”) that may implement brain interface system 102. As shown, system 800 includes a wearable sensor unit 802 and a controller 804. Wearable sensor unit 802 includes a plurality of magnetometers 806-1 through 806-N (collectively “magnetometers 806”, also referred to as optically pumped magnetometer (OPM) modular assemblies as described below) and a magnetic field generator 808. Wearable sensor unit 802 may include additional components (e.g., one or more magnetic field sensors, position sensors, orientation sensors, accelerometers, image recorders, detectors, etc.) as may serve a particular implementation. System 800 may be used in magnetoencephalography (MEG) and/or any other application that measures relatively weak magnetic fields.

Wearable sensor unit 802 is configured to be worn by a user (e.g., on a head of the user). In some examples, wearable sensor unit 802 is portable. In other words, wearable sensor unit 802 may be small and light enough to be easily carried by a user and/or worn by the user while the user moves around and/or otherwise performs daily activities, or may be worn in a magnetically shielded environment which allows for natural user movement as described more fully in U.S. Provisional Patent Application No. 63/076,015, and U.S. Non-Provisional patent application Ser. No. 17/328,235, filed May 24, 2021 and published as US2021/0369166A1, previously incorporated by reference.

Any suitable number of magnetometers 806 may be included in wearable sensor unit 802. For example, wearable sensor unit 802 may include an array of nine, sixteen, twenty-five, or any other suitable plurality of magnetometers 806 as may serve a particular implementation.

Magnetometers 806 may each be implemented by any suitable combination of components configured to be sensitive enough to detect a relatively weak magnetic field (e.g., magnetic fields that come from the brain). For example, each magnetometer may include a light source, a vapor cell such as an alkali metal vapor cell (the terms “cell”, “gas cell”, “vapor cell”, and “vapor gas cell” are used interchangeably herein), a heater for the vapor cell, and a photodetector (e.g., a signal photodiode). Examples of suitable light sources include, but are not limited to, a diode laser (such as a vertical-cavity surface-emitting laser (VCSEL), distributed Bragg reflector laser (DBR), or distributed feedback laser (DFB)), light-emitting diode (LED), lamp, or any other suitable light source. In some embodiments, the light source may include two light sources: a pump light source and a probe light source.

Magnetic field generator 808 may be implemented by one or more components configured to generate one or more compensation magnetic fields that actively shield magnetometers 806 (including respective vapor cells) from ambient background magnetic fields (e.g., the Earth's magnetic field, magnetic fields generated by nearby magnetic objects such as passing vehicles, electrical devices and/or other field generators within an environment of magnetometers 806, and/or magnetic fields generated by other external sources). For example, magnetic field generator 808 may include one or more coils configured to generate compensation magnetic fields in the Z direction, X direction, and/or Y direction (all directions are with respect to one or more planes within which the magnetic field generator 808 is located). The compensation magnetic fields are configured to cancel out, or substantially reduce, ambient background magnetic fields in a magnetic field sensing region with minimal spatial variability.

Controller 804 is configured to interface with (e.g., control an operation of, receive signals from, etc.) magnetometers 806 and the magnetic field generator 808. Controller 804 may also interface with other components that may be included in wearable sensor unit 802.

In some examples, controller 804 is referred to herein as a “single” controller 804. This means that only one controller is used to interface with all of the components of wearable sensor unit 802. For example, controller 804 may be the only controller that interfaces with magnetometers 806 and magnetic field generator 808. It will be recognized, however, that any number of controllers may interface with components of magnetic field measurement system 800 as may suit a particular implementation.

As shown, controller 804 may be communicatively coupled to each of magnetometers 806 and magnetic field generator 808. For example, FIG. 8 shows that controller 804 is communicatively coupled to magnetometer 806-1 by way of communication link 810-1, to magnetometer 806-2 by way of communication link 810-2, to magnetometer 806-N by way of communication link 810-N, and to magnetic field generator 808 by way of communication link 812. In this configuration, controller 804 may interface with magnetometers 806 by way of communication links 810-1 through 810-N (collectively “communication links 810”) and with magnetic field generator 808 by way of communication link 812.

Communication links 810 and communication link 812 may be implemented by any suitable wired connection as may serve a particular implementation. For example, communication links 810 may be implemented by one or more twisted pair cables while communication link 812 may be implemented by one or more coaxial cables. Alternatively, communication links 810 and communication link 812 may both be implemented by one or more twisted pair cables. In some examples, the twisted pair cables may be unshielded.

Controller 804 may be implemented in any suitable manner. For example, controller 804 may be implemented by a field-programmable gate array (FPGA), an application specific integrated circuit (ASIC), a digital signal processor (DSP), a microcontroller, and/or other suitable circuit together with various control circuitry.

In some examples, controller 804 is implemented on one or more printed circuit boards (PCBs) included in a single housing. In cases where controller 804 is implemented on a PCB, the PCB may include various connection interfaces configured to facilitate communication links 810 and 812. For example, the PCB may include one or more twisted pair cable connection interfaces to which one or more twisted pair cables may be connected (e.g., plugged into) and/or one or more coaxial cable connection interfaces to which one or more coaxial cables may be connected (e.g., plugged into).

In some examples, controller 804 may be implemented by or within a computing device.

In some examples, a wearable magnetic field measurement system may include a plurality of optically pumped magnetometer (OPM) modular assemblies, which OPM modular assemblies are enclosed within a housing sized to fit into a headgear (e.g., brain interface system 102) for placement on a head of a user (e.g., human subject). The OPM modular assembly is designed to enclose the dements of the OPM optics, vapor cell, and detectors in a compact arrangement that can be positioned dose to the head of the human subject. The headgear may include an adjustment mechanism used for adjusting the headgear to conform with the human subject's head. These exemplary OPM modular assemblies and systems are described in more detail in U.S. Provisional Patent Application No. 63/170,892, previously incorporated by reference in its entirety.

At least some of the dements of the OPM modular assemblies, systems which can employ the OPM modular assemblies, and methods of making and using the OPM modular assemblies have been disclosed in U.S. Patent Application Publications Nos. 2020/0072916; 2020/0056263; 2020/0025844; 2020/0057116; 2019/0391213; 2020/0088811; 2020/0057115; 2020/0109481; 2020/0123416; 2020/0191883; 2020/0241094; 2020/0256929; 2020/0309873; 2020/0334559; 2020/0341081; 2020/0381128; 2020/0400763; 2021/0011094; 2021/0015385; 2021/0041512; 2021/0041513; 2021/0063510; and 2021/0139742; and U.S. Provisional Patent Application Ser. Nos. 62/689,696; 62/699,596; 62/719,471; 62/719,475; 62/719,928; 62/723,933; 62/732,327; 62/732,791; 62/741,777; 62/743,343; 62/747,924; 62/745,144; 62/752,067; 62/776,895; 62/781,418; 62/796,958; 62/798,209; 62/798,330; 62/804,539; 62/826,045; 62/827,390; 62/836,421; 62/837,574; 62/837,587; 62/842,818; 62/855,820; 62/858,636; 62/860,001; 62/865,049; 62/873,694; 62/874,887; 62/883,399; 62/883,406; 62/888,858; 62/895,197; 62/896,929; 62/898,461; 62/910,248; 62/913,000; 62/926,032; 62/926,043; 62/933,085; 62/960,548; 62/971,132; 63/031,469; 63/052,327; 63/076,015; 63/076,880; 63/080; 248; 63/135,364; 63/136,415; and 63/170,892, all of which are incorporated herein by reference in their entireties.

In some examples, one or more components of brain interface system 102, FIG. 1, (e.g., one or more computing devices) may be configured to be located off the head of the user.

In each of the different brain interface system implementations described herein, the brain activity data may be based on the type of operations performed by the different brain interface system implementations. For example, if brain interface system 102 is implemented by an optical measurement system configured to perform optical-based brain data acquisition operations, the brain activity data may be based on the optical-based brain data acquisition operations. As another example, if brain interface system 102 is implemented by a multimodal measurement system configured to perform optical-based brain data acquisition operations and electrical-based brain data acquisition operations, the brain activity data may be based on the optical-based brain data acquisition operations and the electrical-based brain data acquisition operations. As another example, if brain interface system 102 is implemented by a magnetic field measurement system configured to perform magnetic field-based brain data acquisition operations, the brain activity data may be based on the magnetic field-based brain data acquisition operations.

As mentioned, computing device 104 may be configured to determine a characteristic of a user based on brain activity data output by brain interface system 102. This may be performed in any suitable manner. For example, computing device 104 may use any suitable statistical analysis and/or or other data processing technique to transform the brain activity data into user characteristic data representative of a characteristic of the user.

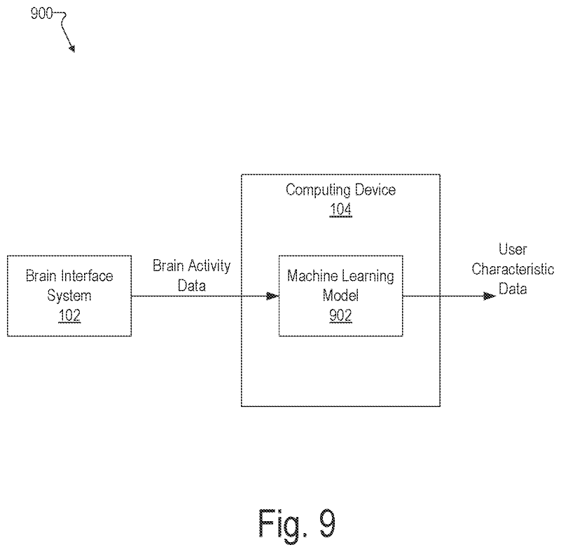

In some examples, computing device 104 may use a machine learning model to determine the characteristic. FIG. 9 shows an illustrative configuration 900 in which computing device 104 is configured to implement a machine learning model 902 to determine a characteristic of a user.

Machine learning model 902 may be configured to perform any suitable machine learning heuristic (also referred to as artificial intelligence heuristic) to input data, which may be in either the time or frequency domains. Machine learning model 902 may accordingly be supervised and/or unsupervised as may serve a particular implementation and may be configured to implement one or more decision tree learning algorithms, association rule learning algorithms, artificial neural network learning algorithms, deep learning algorithms, bitmap algorithms, and/or any other suitable data analysis technique as may serve a particular implementation.

In some examples, machine learning model 902 is implemented by one or more neural networks, such as one or more deep convolutional neural networks (CNN) using internal memories of its respective kernels (filters), recurrent neural networks (RNN), and/or long/short term memory neural networks (LSTM). Machine learning model 902 may be multi-layer. For example, machine learning model 902 may be implemented by a neural network that includes an input layer, one or more hidden layers, and an output layer. Machine learning model 902 may be trained in any suitable manner.

A machine learning model, such as machine learning model 902, may be additionally or alternatively used to perform any of the other operations described herein as being performed by computing device 104. For example, computing device 104 may use a machine learning model to control one or more operations of one or more devices (e.g., brain interface system 102, computing device 104, and/or another computing device) based on user characteristic data. As another example, computing device 104 may be configured to use a machine learning model to generate a predicted future characteristic of the user (e.g., a brain state of the user at a particular time in the future) based on the brain activity data output by brain interface system 102.

The user characteristic determined by computing device 104 may include any attribute of the user as may serve a particular implementation. For example, the characteristic may include a mental state of the user during a particular time period.

Example mental states include, but are not limited to, joy, excitement, relaxation, surprise, fear, stress, anxiety, sadness, anger, disgust, contempt, contentment, calmness, approval, focus, attention, creativity, cognitive assessment, positive or negative reflections/attitude on experiences or the use of objects, etc. Further details on the methods and systems related to a predicted brain state, behavior, preferences, or attitude of the user, and the creation, training, and use of neuromes can be found in U.S. patent application Ser. No. 17/188,298, filed Mar. 1, 2021, issued as U.S. Pat. No. 11,132,625. Exemplary measurement systems and methods using biofeedback for awareness and modulation of mental state are described in more detail in U.S. patent application Ser. No. 16/364,338, filed Mar. 26, 2019, issued as U.S. Pat. No. 11,006,876. Exemplary measurement systems and methods used for detecting and modulating the mental state of a user using entertainment selections, e.g., music, film/video, are described in more detail in U.S. patent application Ser. No. 16/835,972, filed Mar. 31, 2020, issued as U.S. Pat. No. 11,006,878. Exemplary measurement systems and methods used for detecting and modulating the mental state of a user using product formulation from, e.g., beverages, food, selective food/drink ingredients, fragrances, and assessment based on product-elicited brain state measurements are described in more detail in U.S. patent application Ser. No. 16/853,614, filed Apr. 20, 2020, issued as U.S. Pat. No. 11,172,869. Exemplary measurement systems and methods used for detecting and modulating the mental state of a user through awareness of priming effects are described in more detail in U.S. patent application Ser. No. 16/885,596, filed May 28, 2020, published as US2020/0390358A1. These applications and corresponding U.S. patents and publications are incorporated herein by reference in their entirety.

Additionally or alternatively, the characteristic determined by computing device 104 may include a strength of connection between different brain regions of the user. These brain regions may be defined by anatomical constraints or by regions of the brain that function similarly. For example, individual regions or networks of regions may be grouped by function (e.g., an attention network) and/or anatomical connections (e.g., the frontoparietal network). As described herein, these brain regions may be identified and shown together in a manner that a user may readily ascertain a relatively strength of connection between the different brain regions. This, in turn, may allow a user to focus on efforts specifically intended to strengthen particular connections between the brain regions.

Additionally or alternatively, the characteristic determined by computing device 104 may include magnitude of a brain response of the user, power in a neural frequency band of the user, a location of an active brain region of the user, an age of the brain of the user, a health of the brain of the user, an efficiency of the brain of the user, an ability of the user to focus on a particular task, and/or any other attribute as may serve a particular implementation.

Various examples of graphical content that may be presented by computing device 104 will now be described. The various examples of graphical content described herein are merely illustrative of the many different ways in which user characteristic data associated with a user may be presented. Moreover, in the following examples, it is assumed that computing device 104 determines the characteristic of the user by determining a plurality of mental states that the user is in during different periods of time. Similar graphical content may be presented for other types of characteristics.

In some examples, computing device 104 may present the graphical content by presenting one or more graphics representative of the mental states. This may be done in any suitable manner. For example, in situations where computing device 104 determines that the user is in a first mental state during a first period of time and in a second mental state during a second period of time, computing device 104 may present a first graphic associated with the first mental state and representative of an amount of the brain activity while the user is in the first mental state and a second graphic associated with the second mental state and representative of an amount of the brain activity while the user is in the second mental state.

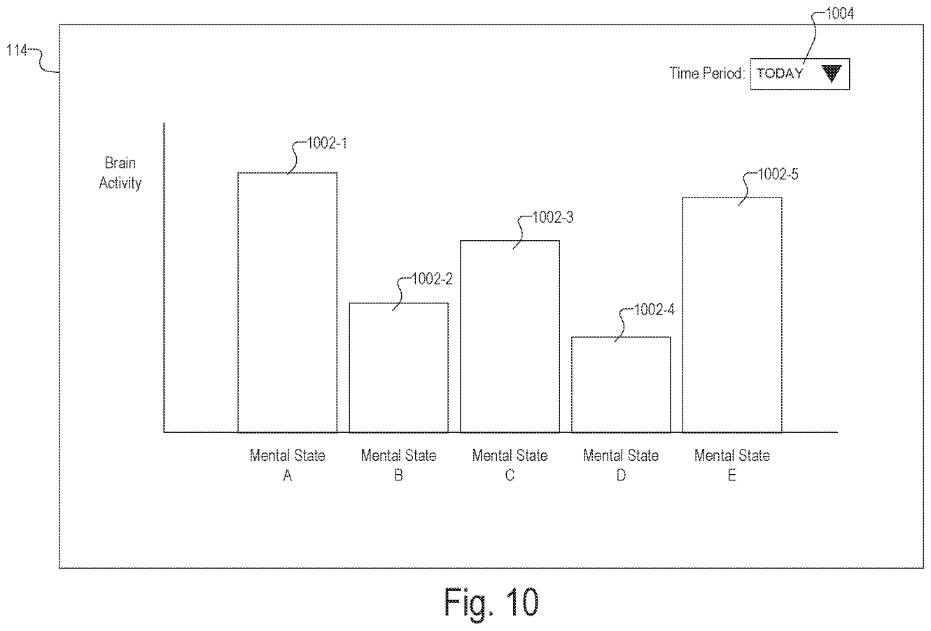

To illustrate, FIG. 10 shows a plurality of graphics 1002-1 through 1002-5 (collectively “graphics 1002”) that may be presented within graphical user interface 114 and that may be representative of an amount of brain activity of the user while the user is in a plurality of different mental states (e.g., mental state A through mental state E) over the course of a user-definable time period 1004. The amount of brain activity of the user corresponding to each mental state may be representative of a cumulative amount of brain activity observed over the course of the time period 1004 for a given mental state, an intensity of the brain activity while the user is in a given mental state, etc., and may be determined in any suitable manner. For example, the amount of brain activity may be derived from blood flow measurements within the brain, etc.

By viewing graphical user interface 114, a user may readily ascertain which mental state uses the most brain activity, which mental state the user was in the most during the time period, and/or any of a number of other characteristics of the brain activity while in the different mental states. For example, as graphic 1002-1 has the highest amplitude in FIG. 10, the user may determine that he or she was in mental state A for the longest amount of time during time period 1004.

As shown, in some examples, the user may select a different time period 1004 to view relative amounts of brain activity for the different time period. Example time periods include an hour, a day, a week, a month, a year, and/or any other suitable amount of time.

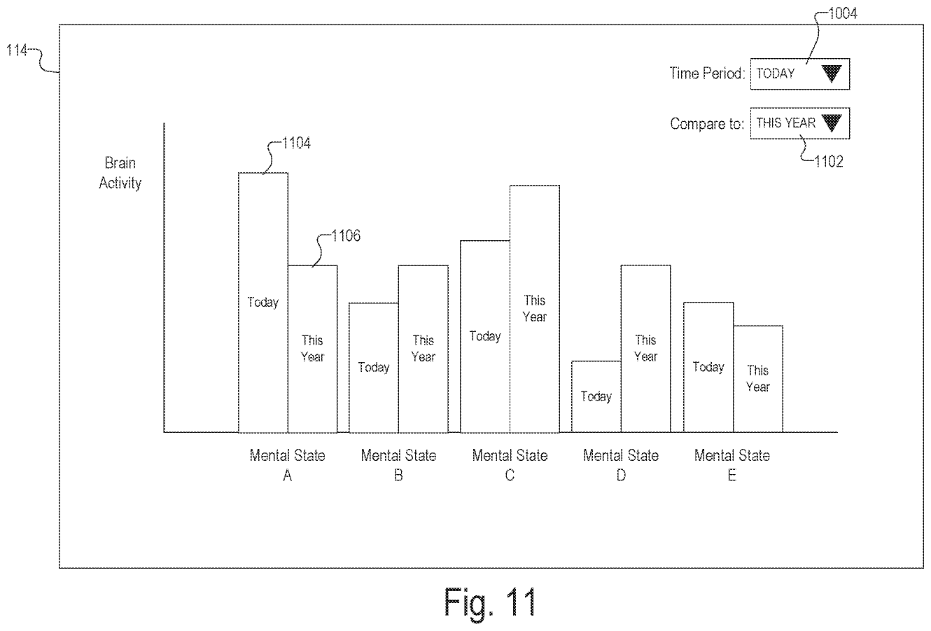

FIG. 11 shows another configuration in which computing device 104 may be configured to provide an option for the user to compare brain activity while in different mental states to a time-based baseline (e.g., another time period). For example, as shown, computing device 104 may present a user-selectable time period 1004 and a user-selectable comparison time period 1102 within graphical user interface 114. In the example of FIG. 11, the user has selected the time period to be “today” and the comparison time period to be “this year”. Accordingly, computing device 104 may present graphics for each mental state corresponding to “today” (e.g., graphic 1104) and “this year” (e.g., graphic 1106). In this manner, the user may readily ascertain how he or she is doing at being within a desired mental state compared to his or her historical tendencies.

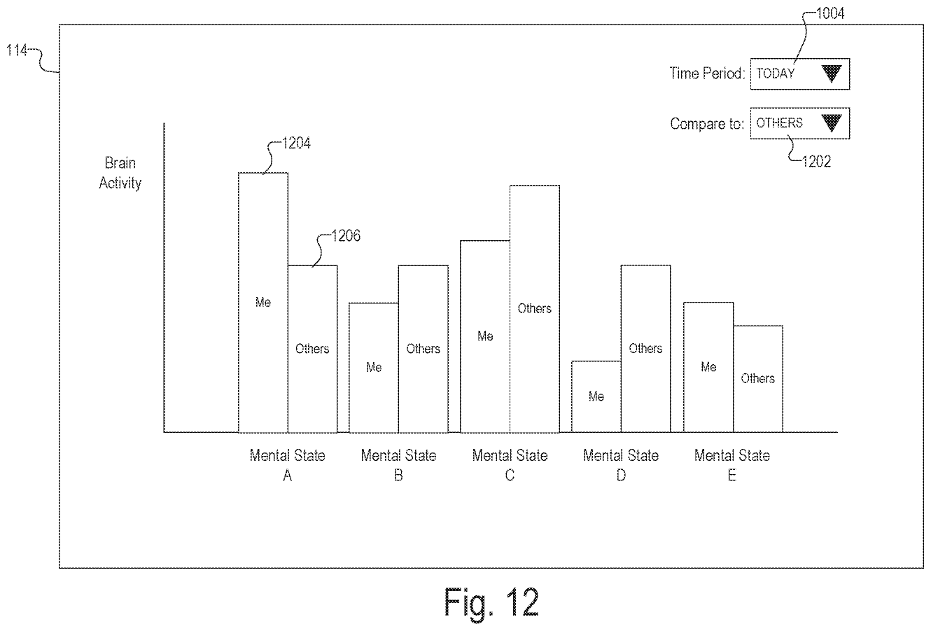

FIG. 12 shows another configuration in which computing device 104 may be configured to provide an option for the user to compare brain activity while in different mental states to one or more other users of brain interface systems. The other users may be filtered to include users having one or more specific characteristics (e.g., gender, age, race, income, profession, etc.).

For example, as shown, computing device 104 may present a user-selectable time period 1004 and a user-selectable comparison 1202 within graphical user interface 114. In the example of FIG. 12, the user has selected the time period to be “today” and the comparison group to be “others”. Accordingly, computing device 104 may present graphics for each mental state corresponding to the user (e.g., graphic 1204) and other people (e.g., graphic 1206). In this manner, the user may readily ascertain how he or she is doing at being within a desired mental state compared to other people.

In some examples, computing device 104 may determine that that the user is in a first mental state during a first time period and that the user is in a second mental state during a second time period. In these examples, computing device 104 may present the graphical content by presenting a first graphic indicating that the user is in the first mental state during the first time period and a second graphic indicating that the user is in the second mental state during the second time period.

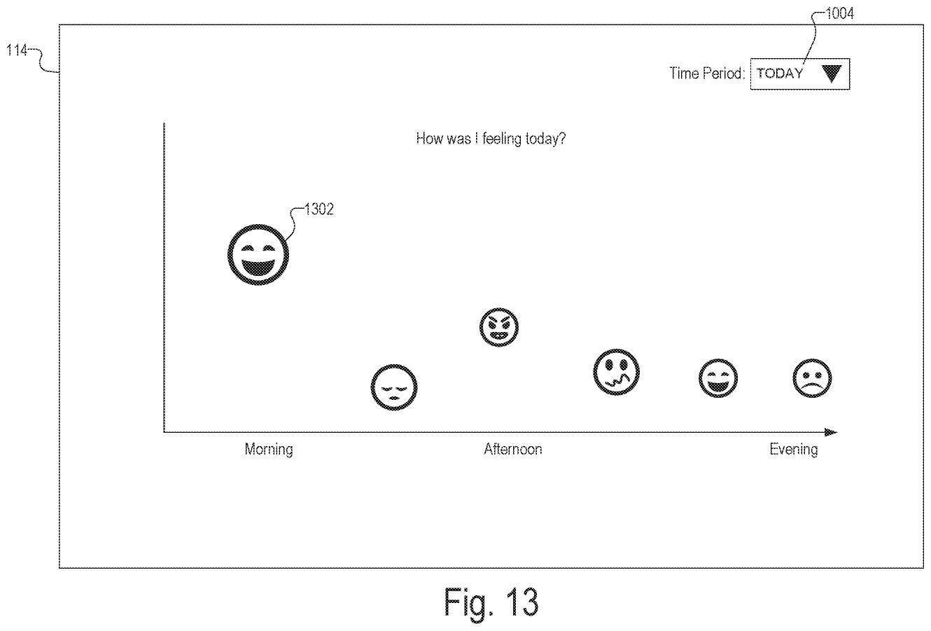

To illustrate, FIG. 13 shows a plurality of emojis (e.g., emoji 1302) displayed along a timeline associated with a user-defined time period 1004 (in this example, a day). In this example, each emoji may represent a different brain state (e.g., happy, sad, tired, confused, angry, etc.). In this manner, the user may readily ascertain his or her brain state at different times throughout the time period.

In some examples, the amount of brain activity while the user is in one or more mental states may correspond to one or more specific regions of a brain of the user. As used herein, a brain region may be defined by anatomical constraints (e.g., a frontoparietal region) an/or by regions of the brain that function similarly (e.g., the attention network). Accordingly, computing device 104 may present graphics associated with the one or more mental states within a depiction of the brain that shows the various regions.

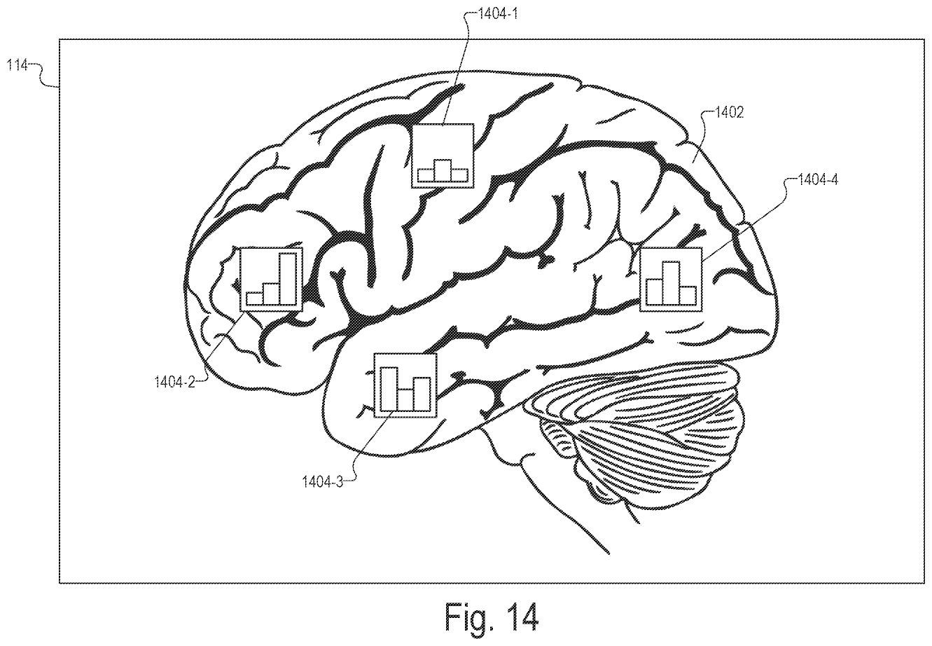

To illustrate, FIG. 14 shows that an image 1402 of a brain may be presented within graphical user interface 114. Graphics 1404-1 through 1404-4 representative of brain activity within certain regions of the brain may be overlaid on top of different regions of the brain as shown in image 1402. The brain activity may be broken out by different mental states as described herein, and as illustrated by the bar graphs shown in each of graphics 1404.

In some examples, computing device 104 may determine a characteristic of the user determining a strength of connection between different brain regions. The strength of connection may represent how well the different brain regions interact with each other. In these examples, computing device 104 may present graphics representative of the different brain regions and graphics representative of the strengths of connection.

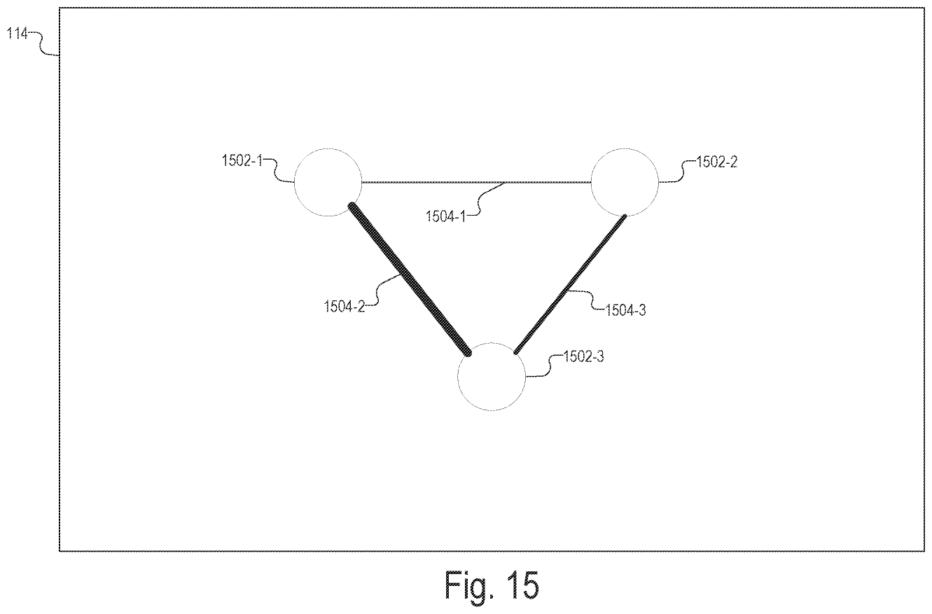

To illustrate, FIG. 15 shows a plurality of nodes 1502-1 through 1502-3 (collectively “nodes 1502”) and edges 1504-1 through 1504-3 (collectively “edges 1504”). Each node 1502 may represent a particular brain region and/or a plurality of brain regions that have been grouped by function and/or anatomical connections. Each edge 1504 may represent a strength of connection between two brain regions. For example, edge 1504-1 may represent a strength of connection between a first brain region represented by node 1502-1 and a second brain region represented by node 1502-2. In some examples, a thickness of edges 1504 may represent relative strength of connection. For example, edge 1504-2 is thicker than edge 1502-1, thereby indicating that the strength of connection between the first brain region and a third brain region represented by node 1502-3 is relatively stronger than the strength of connection between the first and second brain regions.

In some examples, an edge 1504 may be modified (e.g., using animation and/or other graphical effects) to depict how a strength of connection between brain regions changes over time. This may be performed in any suitable manner.

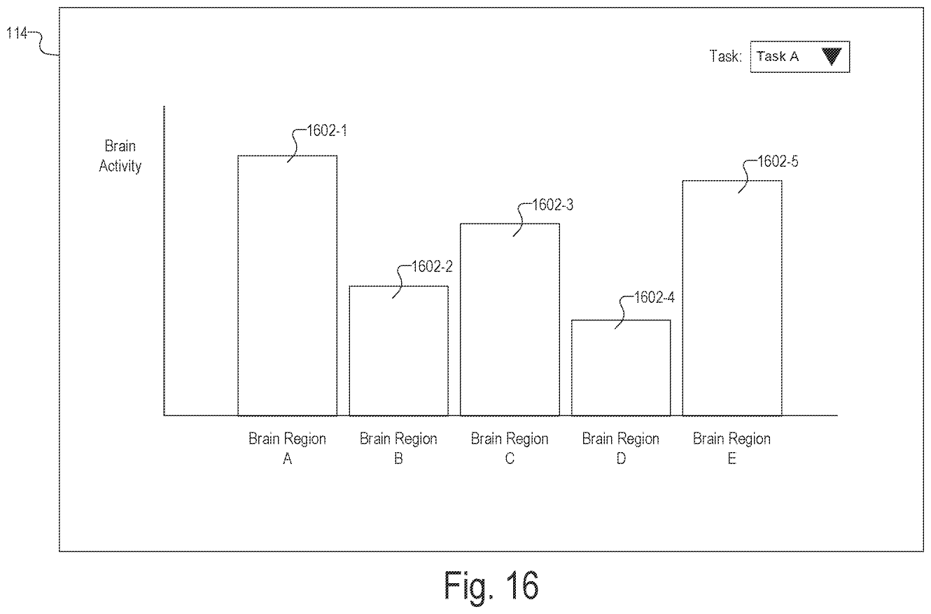

FIG. 16 shows another example of graphical content associated with a user characteristic being presented within graphical user interface 114. In FIG. 16, relative measures of brain activity in different brain regions while a user performs a particular task (e.g., playing a game, watching video content, listening to audio content, working, etc.) are represented by graphics 1602-1 through 1602-5 (collectively “graphics 1602”). In some examples, the task is user-selectable. In some examples, computing device 104 may present, by way of graphical user interface 114, information representative of the task.

Computing device 104 may present graphical content representative of a determined user characteristic in any other way as may serve a particular implementation. For example, computing device 104 may be configured to detect user input representative of a selection by the user of a visual theme. Computing device 104 may be configured to present the graphical content in accordance with the visual theme.

To illustrate, the user may select a visual theme that represents a customized color scale and/or visual style). The visual theme may include icons or other depictions of brain activity as objects (e.g., leaves on a tree, tools in a toolchest, windows and doors in a house, natural elements in a landscape), where features of the objects (e.g., color, size, placement) are related to features of brain activity. Animation could be used to show changes in brain activity over time or between tasks. Brain activity could be visualized in real time or presented after data collection.

As another example, computing device 104 may determine, based on the characteristic of the user, a recommended action for the user to perform and present, by way of graphical user interface 114, content representative of the recommended action. For example, with reference to FIG. 15, computing device 104 may present one or more recommended brain exercises configured to strengthen a particular strength of connection (e.g., the strength of connection represented by edge 1504-1) between brain regions (e.g.; brain regions represented by nodes 1502-1 and 1502-2).

As another example, brain activity data and/or user characteristic data can be presented in a representational or abstract form. For example, if a selected user is measured repeatedly over time, brain data related to task performance, subjective or objective task difficulty, sleep measures or sleep satisfaction, or mood may be displayed to the user. Animation may be used to show changes in brain activity over time while simultaneously displaying another metric of interest.

In some examples, the graphical content may be displayed as a two dimensional (2D) map of brain activity or it could be an interactive three dimensional (3D) visualization of the brain activity. The observers could explore the different regions of activation. In addition, the brain activity could be quantified by regions of interest with a graphical representation of the magnitude of signal in each region shown, e.g.; by a bar.

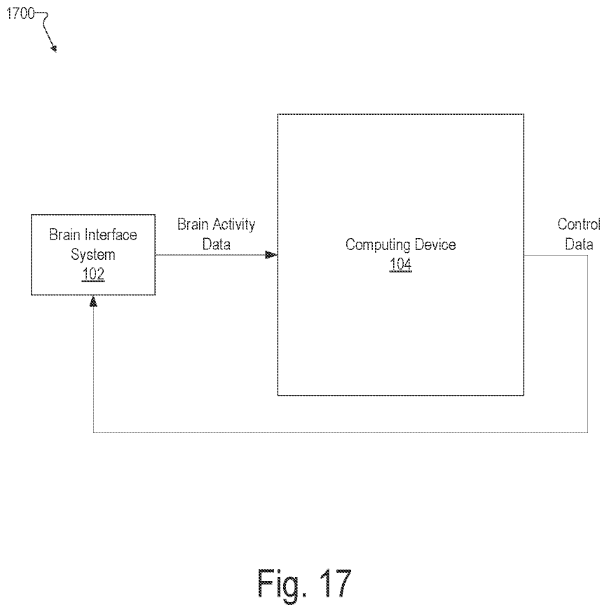

Computing device 104 may perform one or more other types of operations based on a determined user characteristic. For example; FIG. 17 shows a configuration 1700 in which computing device 104 is configured to output, based on a determined user characteristic, control data that may be used to modify an operation of brain interface system 102. The control data may modify the operation of brain interface system 102 in any suitable manner. For example, the control data may modify the operation of brain interface system 102 by adjusting a manner in which the brain activity data is obtained. For example, computing device 104 may transmit a command to brain interface system 102 for brain interface system 102 to reduce a resolution of histogram data output by brain interface system 102 to conserve operating power when computing device 104 determines that the user is in a particular mental state (e.g., when the user is happy and not as interested in knowing how to change his or her mental state, the frequency of brain activity data acquisition may be reduced).

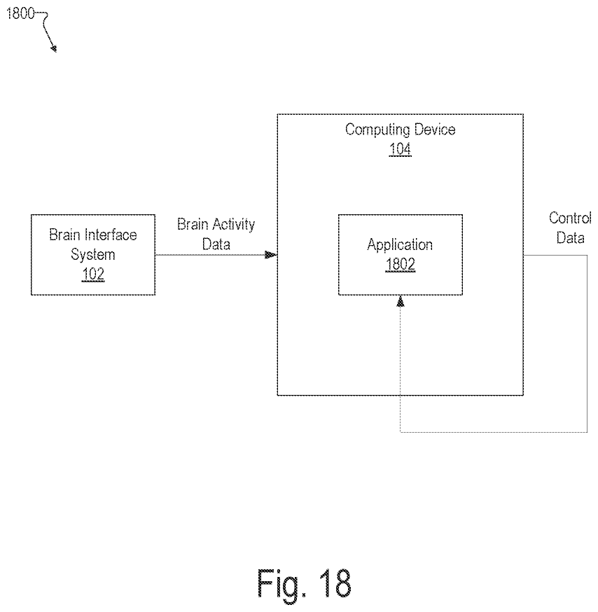

As another example, FIG. 18 shows a configuration 1800 in which computing device 104 is configured to output, based on a determined user characteristic, control data that may be used to modify an operation of an application 1802 (e.g., an electronic game, an electronic learning session, etc.) being executed by computing device 104. For example, based on a particular mental state, a difficult level of application 1802 may be modified.

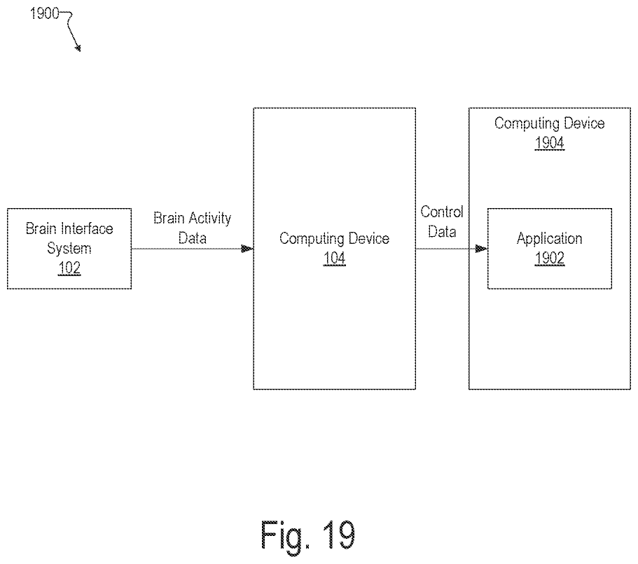

As another example, FIG. 19 shows a configuration 1900 in which computing device 104 is configured to output, based on a determined user characteristic, control data that may be used to modify an operation of an application 1902 (e.g., an electronic game, an electronic learning session, etc.) being executed by another computing device 1904.

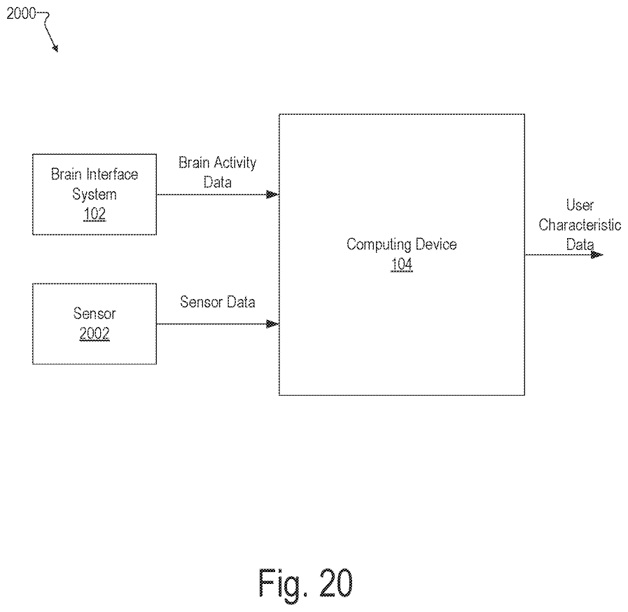

FIG. 20 shows an illustrative configuration 2000 in which computing device 104 is configured to access both brain activity data and sensor data output by a sensor 2002. In this example, computing device 104 may be configured to determine the characteristic of the user based on both the brain activity data and the sensor data.

Sensor 2002 may be implemented in any suitable manner. For example, sensor 2002 may be implemented by one or more sensors that perform eye tracking, electrodermal activity (EDA)/conductance, pupillometry, heart rate, heart rate variability, and/or pulse oximetry. Additionally or alternatively, sensor 2002 may be implemented by one or more microphones configured to detect ambient sound of the user, one or more inertial motion units (IMUS) configured to detect movement by the user, etc. In some examples, the sensor data may be presented within graphical user interface 114 together with any of the other graphical content described herein. An example of this is described in U.S. patent application Ser. No. 17/550,387, filed Dec. 14, 2021 and incorporated herein by reference in its entirety.

In some examples, computing device 104 may be configured to predict, based on the brain activity data, a future characteristic of the user and present, by way of graphical user interface 114, graphical content representative of the future characteristic. For example, based on the brain activity data, computing device 104 may predict that the user will succeed in a particular area of study (e.g., in college). Computing device 104 may accordingly present the particular area of study within graphical user interface 114 and, in some examples, various recommended actions that the user may take to enhance his or her chances of success in the particular area of study.

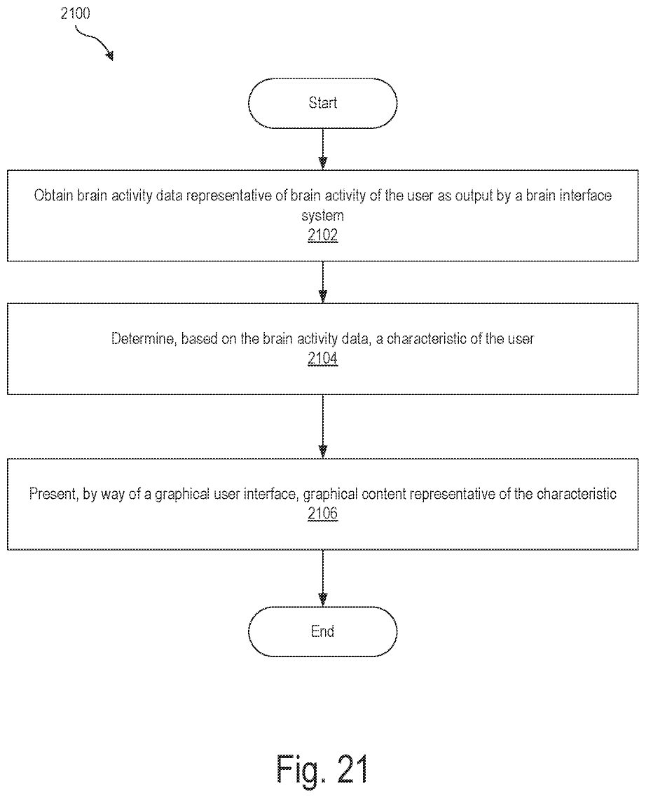

FIG. 21 illustrates an exemplary method 2100. While FIG. 21 illustrates exemplary operations according to one embodiment, other embodiments may omit, add to, reorder, and/or modify any of the operations shown in FIG. 21. One or more of the operations shown in FIG. 21 may be performed by computing device 104 and/or any implementation thereof. Each of the operations illustrated in FIG. 21 may be performed in any suitable manner.

At operation 2102, a computing device may obtain brain activity data representative of brain activity of the user as output by a brain interface system.

At operation 2104, the computing device may determine, based on the brain activity data, a characteristic of the user.

At operation 2106, the computing device may present, by way of a graphical user interface, graphical content representative of the characteristic.

In some examples, a non-transitory computer-readable medium storing computer-readable instructions may be provided in accordance with the principles described herein. The instructions, when executed by a processor of a computing device, may direct the processor and/or computing device to perform one or more operations, including one or more of the operations described herein. Such instructions may be stored and/or transmitted using any of a variety of known computer-readable media.

A non-transitory computer-readable medium as referred to herein may include any non-transitory storage medium that participates in providing data (e.g., instructions) that may be read and/or executed by a computing device (e.g., by a processor of a computing device). For example, a non-transitory computer-readable medium may include, but is not limited to, any combination of non-volatile storage media and/or volatile storage media. Exemplary non-volatile storage media include, but are not limited to, read-only memory, flash memory, a solid-state drive, a magnetic storage device (e.g., a hard disk, a floppy disk, magnetic tape, etc.), ferroelectric random-access memory (“RAM”), and an optical disc (e.g., a compact disc, a digital video disc, a Blu-ray disc, etc.). Exemplary volatile storage media include, but are not limited to, RAM (e.g., dynamic RAM).

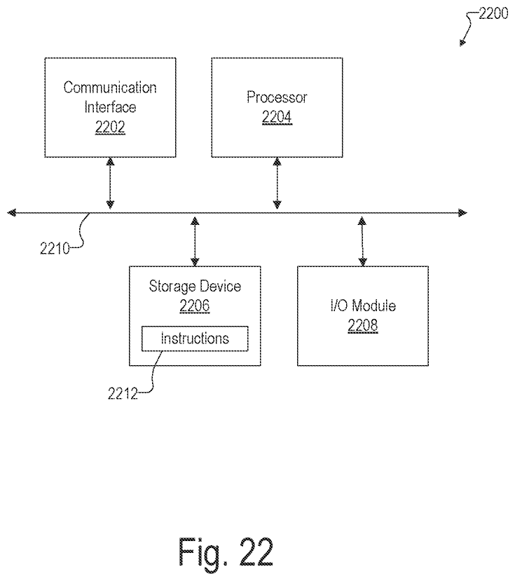

FIG. 22 illustrates an exemplary computing device 2200 that may be specifically configured to perform one or more of the processes described herein. Any of the systems, units, computing devices, and/or other components described herein may be implemented by computing device 2200.

As shown in FIG. 22, computing device 2200 may include a communication interface 2202, a processor 2204, a storage device 2206, and an input/output (“I/O”) module 2208 communicatively connected one to another via a communication infrastructure 2210. While an exemplary computing device 2200 is shown in FIG. 22, the components illustrated in FIG. 22 are not intended to be limiting. Additional or alternative components may be used in other embodiments. Components of computing device 2200 shown in FIG. 22 will now be described in additional detail.

Communication interface 2202 may be configured to communicate with one or more computing devices. Examples of communication interface 2202 include, without limitation, a wired network interface (such as a network interface card), a wireless network interface (such as a wireless network interface card), a modem, an audio/video connection, and any other suitable interface.

Processor 2204 generally represents any type or form of processing unit capable of processing data and/or interpreting, executing, and/or directing execution of one or more of the instructions, processes, and/or operations described herein. Processor 2204 may perform operations by executing computer-executable instructions 2212 (e.g., an application, software, code, and/or other executable data instance) stored in storage device 2206.

Storage device 2206 may include one or more data storage media, devices, or configurations and may employ any type, form, and combination of data storage media and/or device. For example, storage device 2206 may include, but is not limited to, any combination of the non-volatile media and/or volatile media described herein. Electronic data, including data described herein, may be temporarily and/or permanently stored in storage device 2206. For example, data representative of computer-executable instructions 2212 configured to direct processor 2204 to perform any of the operations described herein may be stored within storage device 2206. In some examples, data may be arranged in one or more databases residing within storage device 2206.

I/O module 2208 may include one or more I/O modules configured to receive user input and provide user output. I/O module 2208 may include any hardware, firmware, software, or combination thereof supportive of input and output capabilities. For example, I/O module 2208 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 2208 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 2208 is configured to provide graphical data to a display for presentation to a user. The graphical data may be representative of one or more graphical user interfaces and/or any other graphical content as may serve a particular implementation.

An illustrative system includes a brain interface system configured to be worn by a user and to output brain activity data representative of brain activity of the user and a computing device configured to obtain the brain activity data, determine, based on the brain activity data, a characteristic of the user, and present, by way of a graphical user interface, graphical content representative of the characteristic.

An illustrative apparatus includes a memory storing instructions and a processor communicatively coupled to the memory and configured to execute the instructions to: obtain brain activity data representative of brain activity of the user as output by a brain interface system; determine, based on the brain activity data, a characteristic of the user; and present, by way of a graphical user interface, graphical content representative of the characteristic.

An illustrative method includes obtaining, by a computing device, brain activity data representative of brain activity of the user as output by a brain interface system; determining, by the computing device based on the brain activity data, a characteristic of the user; and presenting, by the computing device by way of a graphical user interface, graphical content representative of the characteristic.

An illustrative non-transitory computer-readable medium storing instructions that, when executed, direct a processor of a computing device to: obtain brain activity data representative of brain activity of the user as output by a brain interface system; determine, based on the brain activity data, a characteristic of the user; and present, by way of a graphical user interface, graphical content representative of the characteristic.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A system comprising:
a brain interface system configured to be worn by a user and to output brain activity data representative of brain activity of the user; and
a computing device configured to
obtain the brain activity data,
determine, based on the brain activity data, a characteristic of the user, and
present, by way of a graphical user interface; graphical content representative of the characteristic.

2. The system of claim 1, wherein the brain interface system comprises an optical measurement system configured to perform optical-based brain data acquisition operations, the brain activity data based on the optical-based brain data acquisition operations.

3. The system of claim 2, wherein the optical measurement system comprises:
a wearable assembly configured to be worn by the user and comprising:
a plurality of light sources each configured to emit light directed at a brain of the user, and
a plurality of detectors configured to detect arrival times for photons of the light after the light is scattered by the brain, the brain activity data based on the arrival times.

4. The system of claim 3, wherein the detectors each comprise a plurality of single-photon avalanche diode (SPAR) circuits.

5. The system of claim 3, wherein the wearable assembly further comprises:
a first module comprising a first light source included in the plurality of light sources and a first set of detectors included in the plurality of detectors; and
a second module physically distinct from the first module and comprising a second light source included in the plurality of light sources and a second set of detectors included in the plurality of detectors.

6. The system of claim 5, wherein the first and second modules are configured to be removably attached to the wearable assembly.

7. The system of claim 1, wherein the brain interface system comprises a multimodal measurement system configured to perform optical-based brain data acquisition operations and electrical-based brain data acquisition operations, the brain activity data based on the optical-based brain data acquisition operations and the electrical-based brain data acquisition operations.

8. The system of claim 7, wherein the multimodal measurement system comprises:
a wearable assembly configured to be worn by the user and comprising:
a plurality of light sources each configured to emit light directed at a brain of the user,
a plurality of detectors configured to detect arrival e for photons of the light after the light is scattered by the brain, and
a plurality of electrodes configured to be external to the user and detect electrical activity of the brain, the brain activity based on the arrival times and the electrical activity.

9. The system of claim 8, wherein the wearable assembly further comprises:
a first module comprising a first light source included in the plurality of light sources and a first set of detectors included in the plurality of detectors; and
a second module physically distinct from the first module and comprising a second light source included in the plurality of light sources and a second set of detectors included in the plurality of detectors.

10. The system of claim 9, wherein the plurality of electrodes comprises a first electrode on a surface of the first module and a second electrode on a surface of the second module.

11. The system of claim 10, wherein the first electrode surrounds the first light source on the surface of the first module.

12. The system of claim 1, wherein the obtaining the brain activity data, the determining the characteristic, and the presenting the graphical content are performed in substantially real time while the brain interface system outputs the brain activity data.

13. The system of claim 1, wherein the obtaining the brain activity data comprises receiving the brain activity data from the brain interface system by way of one or more of a wired connection or a wireless connection.

14. The system of claim 1, wherein the computing device is included in the brain interface system.

15. The system of claim 1, wherein the determining the characteristic of the user comprises determining one or more mental states of the user during one or more time periods.

16. The system of claim 15, wherein the presenting the graphical content comprises presenting one or more graphics representative of the one or more mental states.

17. The system of claim 16, wherein:
the one or more mental states comprises a first mental state and a second mental state;
the presenting the one or more graphics comprises:
presenting a first graphic associated with the first mental state and representative of an amount of the brain activity while the user is in the first mental state; and
presenting a second graphic associated with the second mental state and representative of an amount of the brain activity while the user is in the second mental state.

18. The system of claim 17, wherein:
the amount of brain activity while the user is in the first mental state and the amount of brain activity while the user is in the second mental state correspond to a specific region of a brain of the user; and
the presenting the first and second graphics comprises presenting the first and second graphics within a depiction of the specific region of the brain.

19. The system of claim 17, wherein:
the determining the one or more mental states comprises determining that the user is in a first mental state during a first time period and that the user is in a second mental state during a second time period; and
the presenting the graphical content comprises presenting a first graphic indicating that the user is in the first mental state during the first time period and a second graphic indicating that the user is in the second mental state during the second time period.

20. The system of claim 1, wherein:
the determining the characteristic of the user comprises determining a strength of connection between a first brain region of the user and a second brain region of the user; and
the presenting comprises presenting a first graphic representative of the first brain region, a second graphic representative of the second brain region, and a third graphic representative of the strength of connection.

21. The system of claim 20, wherein the presenting further comprises modifying the third graphic to depict how the strength of connection changes over time.

22. The system of claim 1, wherein:
the computing device is further configured to detect user input representative of a selection by the user of a visual theme; and
the presenting the graphical content comprises presenting the graphical content in accordance with the visual theme.

23. The system of claim 1, wherein:
the computing device is further configured to obtain sensor data representative of a sensed attribute of the user; and
the determining of the characteristic of the user is further based on the sensor data.

24. The system of claim 1, wherein the determining the characteristic of the user comprises determining one or more of a magnitude of a brain response of the user, power in a neural frequency band, a location of an active brain region of the user, an age of the brain, a health of the brain, an efficiency of the brain, or an ability of the user to focus on a particular task.

25. The system of claim 1, wherein the computing device is further configured to:
determine, based on the characteristic of the user, a recommended action for the user to perform; and
present, by way of the graphical user interface, content representative of the recommended action.

26. The system of claim 1, wherein:
the computing device is further configured to determine a task being performed during a time period that corresponds to the brain activity data; and
the presenting further comprises presenting, by way of the graphical user interface, information representative of the task.

27. The system of claim 1, wherein the computing device is further configured to modify, based on the characteristic of the user, an operation of the brain interface system.

28. The system of claim 27, wherein the modifying the operation comprises adjusting a manner in which the brain activity data is obtained.

29. The system of claim 1, wherein the computing device is further configured to modify, based on the characteristic of the user, an attribute of an application being executed by at least one of the computing device or a different computing device.

30. The system of claim 1, wherein the computing device is further configured to:
predict, based on the brain activity data, a future characteristic of the user; and
present, by way of a graphical user interface, graphical content representative of the future characteristic.

31. A system comprising:
a memory storing instructions; and
a processor communicatively coupled to the memory and configured to execute the instructions to:
obtain brain activity data representative of brain activity of a user as output by a brain interface system;
determine, based on the brain activity data, a characteristic of the user; and
present, by way of a graphical user interface, graphical content representative of the characteristic.

32. The system of claim 31, wherein the determining the characteristic of the user comprises determining one or more mental states of the user during one or more time periods.

33. The system of claim 32, wherein the presenting the graphical content comprises presenting one or more graphics representative of the one or more mental states.

34. The system of claim 33, wherein:
the one or more mental states comprises a first mental state and a second mental state;
the presenting the one or more graphics comprises:
presenting a first graphic associated with the first mental state and representative of an amount of the brain activity while the user is in the first mental state; and
presenting a second graphic associated with the second mental state and representative of an amount of the brain activity while the user is in the second mental state.

35. The system of claim 34, wherein:
the amount of brain activity while the user is in the first mental state and the amount of brain activity while the user is in the second mental state correspond to a specific region of a brain of the user; and
the presenting the first and second graphics comprises presenting the first and second graphics within a depiction of the specific region of the brain.

36. The system of claim 34, wherein:
the determining the one or more mental states comprises determining that the user is in a first mental state during a first time period and that the user is in a second mental state during a second time period; and
the presenting the graphical content comprises presenting a first graphic indicating that the user is in the first mental state during the first time period and a second graphic indicating that the user is in the second mental state during the second time period.

37. The system of claim 31, wherein:
the determining the characteristic of the user comprises determining a strength of connection between a first brain region of the user and a second brain region of the user; and
the presenting comprises presenting a first graphic representative of the first brain region, a second graphic representative of the second brain region, and a third graphic representative of the strength of connection.

38. The system of claim 37, wherein the presenting further comprises modifying the third graphic to depict how the strength of connection changes over time.

39. The system of claim 31, wherein:
the processor is further configured to execute the instructions to detect user input representative of a selection by the user of a visual theme; and
the presenting the graphical content comprises presenting the graphical content in accordance with the visual theme.

40. The system of claim 31, wherein:
the processor is further configured to execute the instructions to obtain sensor data representative of a sensed attribute of the user; and
the determining of the characteristic of the user is further based on the sensor data.

41. The system of claim 31, wherein the determining the characteristic of the user comprises determining one or more of a magnitude of a brain response of the user, power in a neural frequency band, a location of an active brain region of the user, an age of the brain, a health of the brain, an efficiency of the brain, or an ability of the user to focus on a particular task.

42. The system of claim 31, wherein the processor is further configured to execute the instructions to:
determine, based on the characteristic of the user, a recommended action for the user to perform; and
present, by way of the graphical user interface, content representative of the recommended action.

43. The system of claim 31, wherein:
the processor is further configured to execute the instructions to determine a task being performed during a time period that corresponds to the brain activity data; and
the presenting further comprises presenting, by way of the graphical user interface, information representative of the task.

44. The system of claim 31, wherein the processor is further configured to execute the instructions to modify, based on the characteristic of the user, an operation of the brain interface system.

45. The system of claim 44, wherein the modifying the operation comprises adjusting a manner in which the brain activity data is obtained.

46. The system of claim 31, wherein the processor is further configured to execute the instructions to modify, based on the characteristic of the user, an attribute of an application being executed by a computing device.

47. The system of claim 31, wherein the processor is further configured to execute the instructions to:
predict, based on the brain activity data, a future characteristic of the user; and
present, by way of a graphical user interface, graphical content representative of the future characteristic.

48. A method comprising:
obtaining, by a computing device, brain activity data representative of brain activity of a user as output by a brain interface system;
determining, by the computing device based on the brain activity data, a characteristic of the user; and
presenting, by the computing device by way of a graphical user interface, graphical content representative of the characteristic.

49. A non-transitory computer-readable medium storing instructions that, when executed, direct a processor of a computing device to:
obtain brain activity data representative of brain activity of a user as output by a brain interface system;
determine, based on the brain activity data, a characteristic of the user; and
present, by way of a graphical user interface, graphical content representative of the characteristic.
